(19) United States
(12) Patent Application Publication
(10) Pub. No.: US 2008/0233159 A1
(43) Pub. Date: Sep. 25, 2008

(54) Home-use insecticide

(75) Inventors: Yoshio Katsuda, Nishinomiya-shi (JP); Toshio Takahashi, Takrazuka-shi (JP)

Correspondence Address: OLIFF & BERRIDGE, PLC, P.O. BOX 320850, ALEXANDRIA, VA, 22320-4850, US

(73) Assignee: DAINIHON JOCHUGIKU CO., LTD., OSAKA-SHI (JP)

(21) Appl. No.: 11/798,463

(22) Filed: May 14, 2007

(30) Foreign Application Priority Data
Mar. 22, 2007 (JP) 2007-074304

(51) Int. Cl. A01N 25/00; A01P 15/00; C07C 69/747

(52) U.S. Cl. 424/405; 560/124

(57) ABSTRACT

The present invention provides a home-use insecticide formulated as an insect knockdown agent and insect killing agent that contains a useful pyrethroid compound having superior residual efficacy. The home-use insecticide contains (d)-2-methyl-4-oxo-3-(2-propynyl)-2-cyclopenten-1-yl (d)-trans-2,2-dimethyl-3-(2,2-dichlorovinyl)cyclopropanecarboxylate as an active ingredient thereof.

### BACKGROUND OF THE INVENTION

1. Field of the Invention

The present invention relates to an improvement of home-use insecticide.

2. Description of the Related Art

The active ingredients of home-use insecticides, and particularly aerosol insecticides, targeted for use against flying insects such as flies and mosquitoes currently consist mainly of the combined use of a knockdown agent in the form of d-T80-phthalthrin and a killing agent in the form of d-T80-resmethrin. The d-T80-phthalthrin demonstrates outstanding knockdown effects against flying insects as compared with various other pyrethroid compounds, and although there is currently no knockdown agent that is more useful than this compound, it is required to be used in combination with a killing agent due to its lack of lethal effects.

On the other hand, although imiprothrin is a typical example of a knockdown agent for use against crawling insects such as cockroaches, its lethal effects are not necessarily satisfactory and since it also has the shortcoming of a lack of residual efficacy, it is required to be used in combination with other agents having ample lethal effects and residual efficacy.

In this manner, it has thus far been thought that pyrethroid compounds generally having high knockdown effects are inferior in terms of lethal effects and residual efficacy, while conversely, pyrethroid compounds having superior lethal effects and residual efficacy have low knockdown effects. One of the reasons for this is that, target insects rapidly knocked down at extremely low levels of a knockdown agent are presumed to be unable to ingest an adequate amount of insecticide components to cause death due to subsequent lowering of the activity of insecticide components on the nervous system.

Thus, although there has been a strong desire for the development of an insecticide component for flying insects having superior knockdown effects and lethal effects, and an insecticide component for crawling insects having superior knockdown effects, lethal effects and residual efficacy, this has previously been thought to be difficult based on the aforementioned preconception.

2-Methyl-4-oxo-3-(2-propynyl)-2-cyclopenten-1-yl 2,2-dimethyl-3-(2,2-dichlorovinyl)cyclopropane carboxylate has eight isomers based on two asymmetric carbons of the acid portion and one asymmetric carbon of the alcohol portion. Although Japanese Patent Publication No. Sho 61-57820 (Patent Document 1) discloses that a mixture of these isomers has high knockdown effects, there are no specific examples of the (d)-2-methyl-4-oxo-3-(2-propynyl)-2-cyclopenten-1-yl (d)-trans-2,2-dimethyl-3-(2,2-dichlorovinyl)cyclopropane carboxylate (referred to as the subject compound) employed in the present invention indicated. In addition, although lethal effects and residual efficacy are important factors for insecticides, there is no evidence whatsoever present in Patent Document 1 from the viewpoints of rapid action, lethal effects and residual efficacy regarding the subject compound as a matter of course or isomer mixtures.

However, Pestic. Sci. 6, 537 (1975) (Non-Patent Document 1) describes that, in the practically used permethrin (phenoxybenzyl ester of 2,2-dimethyl-3-(2,2-dichlorovinyl)cyclopropanecarboxylic acid in which the acid portion is the same as that of the subject compound), an ester of the d-cis form acid demonstrated higher insecticidal efficacy against house flies than an ester of the d-trans form acid. On the other hand, Agr. Biol. Chem. 37, 2681 (1973) (Non-Patent Document 2) reports that in a similarly practically used phenothrin (ester in which the alcohol portion is the same as that of permethrin and the acid portion is 2,2-dimethyl-3-(2-methyl-1-propenyl)cyclopropanecarboxylic acid), an ester of the d-trans form acid conversely demonstrated superior lethal efficacy as compared with an ester of the d-cis form acid.

In addition, Jpn. J. Sanit. Zool., Vol. 38, 219 (1987) (Non-Patent Document 3) relates to prallethrin (2-methyl-4-oxo-3-(2-propynyl)-2-cyclopenten-1-yl 2,2-dimethyl-3-(2-methyl-1-propenyl)cyclopropane carboxylate) in which the alcohol portion is the same as the subject compound while the acid portion is the same as the phenothrin of Non-Patent Document 2. The Non-Patent Document 3 discloses that similar to the aforementioned phenothrin, prallethrin in which the acid portion is an ester of the d-trans form demonstrates a higher level of insecticidal activity than an ester of the d-cis form acid.

In this manner, on the basis of Non-Patent Documents 1 to 3, it is predicted to be generally accepted that, in contrast to esters of d-trans form acids demonstrating higher insecticidal efficacy than esters of d-cis form acids among esters in which the acid portion is 2,2-dimethyl-3-(2-methyl-1-propenyl)cyclopropanecarboxylic acid, among esters of 2,2-dimethyl-3-(2,2-dichlorovinyl)cyclopropanecarboxylic acids, including the subject compound, esters of d-cis form acids are conversely superior. Thus, as a result of the inventors of the present invention actually conducting tests on each isomer of the subject compound itself without being preoccupied with generally accepted theories, it was found that, contrary to expectations, esters of the d-trans form demonstrate higher insecticidal activity, thereby leading to selection of the subject compound.

### SUMMARY OF THE INVENTION

An object of the present invention is to focus on 2-methyl-4-oxo-3-(2-propynyl)-2-cyclopenten-1-yl 2,2-dimethyl-3-(2,2-dichlorovinyl)cyclopropanecarboxylate, and together with selecting a particularly useful isomer, develop a home-use insecticide that contains that isomer as an insect knockdown agent, insect killing agent and high residual efficacy component thereof.

The inventors of the present invention conducted extensive studies to solve the aforementioned problems, the results of which led to the completion of the present invention. Namely, the present invention employs the constitution indicated below.

(1) A home-use insecticide comprising (d)-2-methyl-4-oxo-3-(2-propynyl)-2-cyclopenten-1-yl (d)-trans-2,2-dimethyl-3-(2,2-dichlorovinyl)cyclopropanecarboxylate as an insect knockdown agent and insect killing agent.
(2) The home-use insecticide described in (1) above, wherein the active ingredient is effective as both an insect knockdown agent and an insect killing agent.
(3) The home-use insecticide according to (1) above, wherein the insecticide is in the form of an aerosol.

### DESCRIPTION OF THE PREFERRED EMBODIMENTS

The inventors of the present invention conducted extensive studies on eight isomers of 2-methyl-4-oxo-3-(2-propynyl)-2-cyclopenten-1-yl 2,2-dimethyl-3-(2,2-dichlorovinyl)cyclopropanecarboxylate, leading to confirmation that one of those isomers, namely the subject compound indicated below, is able to demonstrate extraordinarily superior and useful effects as an insect knockdown agent and insect killing agent useful for a home-use insecticide:

(d)-2-methyl-4-oxo-3-(2-propynyl)-2-cyclopenten-1-yl (d)-trans-2,2-dimethyl-3-(2,2-dichlorovinyl)cyclopropanecarboxylate.

As was explained regarding Non-Patent Documents 1, 2 and 3 in the section on the Description of the Related Art, although it was initially predicted that esters in which the acid portion is in the d-cis form have superior insecticidal efficacy than esters in which the acid portion is in the d-trans form as in the subject compound, as a result of testing, it was surprisingly verified that the subject compound has outstanding insecticidal efficacy.

Although the home-use insecticide of the present invention can be applied to various preparations such as aerosols, liquids (including oily agents, emulsions and water-soluble agents), mosquito incenses, mosquito capturing mats, mosquito capturing liquids, powders or granules, and sprays including aerosols are the most practical.

When preparing the aerosol, a solvent, and a synergist, other active ingredient or supplementary ingredient and so forth, are incorporated into the subject compound in accordance with ordinary methods to prepare an undiluted aerosol liquid followed by filling this into an aerosol container, filling the container with a propellant and pressurizing.

The incorporated amount of the subject compound in the aerosol insecticide is suitably about 0.02 to 1.0% by weight, and an oily or aqueous undiluted aerosol liquid can be prepared.

Although kerosene can be used easily as an oily solvent, this may be used in combination with, an ester-based solvent (such as isopropyl myristate), a glycol ether-based solvent, a ketone-based solvent or an aromatic hydrocarbon-based solvent. In addition, in the preparation of an aqueous undiluted aerosol liquid, a surfactant (such as polyoxyethylene alkyl ethers, polyoxyethylene sorbitan fatty acid esters or glycerin fatty acid esters), a dispersant or a cosolvent may be suitably incorporated with water. In addition, supplementary ingredients such as stabilizers (such as BHT or BHA), rust preventives (such as sodium benzoate or ammonium citrate) or fragrances are added as necessary.

A synergist such as N-(2-ethylhexyl)-bicyclo[2.2.1]-hept-5-ene-2,3-dicarboxylmide, N-(2-ethylhexyl)-1-isopropyl-bicyclo[2.2.2]-oct-5-ene-2,3-dicarboxylmide or piperonyl butoxide may be added to the undiluted aerosol liquid.

Moreover, multipurpose compositions having superior efficacy are obtained by incorporating other insecticide and insect control components including conventional pyrethroid-based insecticides such as pyrethrin, allethrin, prallethrin, furamethrin, imiprothrin, phenothrin or permethrin, organic silicon-based compounds such as silafluofen, antibacterial and anti-mold components such as hinokitiol or isopropylmethyl phenol, myiticides such as 5-chloro-2-trifluoromethanesulfonamide methyl benzoate, repellents such as diethyltoluamide, deodorizers and fragrances, and synergistic effects can be expected to be adequately demonstrated among the components.

Examples of propellants include liquefied petroleum gas, dimethyl ether and compressed gas (such as nitrogen or carbon dioxide).

Although there are no particular limitations on the filling ratio between the undiluted aerosol liquid and the propellant, it is suitably about 20/80 to 65/35 (by volume).

An aerosol spraying apparatus for spraying the aerosol insecticide of the present invention is composed of, for example, an aerosol container filled with an aerosol insecticide, a valve, and an actuator attached to the stem of the valve, and a spray button containing a spraying port and the like is installed on the actuator. The aerosol container is typically a 180 mL, 300 mL or 45 mL can, and when filling with the aerosol insecticide, the internal pressure of the container is preferably set to about 0.3 to 0.6 MPa in consideration of dispersibility of the sprayed particles.

The home-use insecticide of the present invention can be applied to mosquitoes including infectious disease-carrying mosquitoes such as southern house mosquitoes, Aedes aegypti mosquitoes, Anopheles mosquitoes, Aedes albopictus mosquitoes and common house mosquitoes, flying insects such as flies, shoo flies, moth flies, midges and gnats, crawling insects such as cockroaches and ants, as well as indoor dust mites, clothing-damaging insects such as gypsy moths, Indian meal moths and carpet beetles, stored grain-damaging insects such as boll weevils, and various other harmful insects such as aphids, delphacids, stinkbugs and centipedes, and high insecticidal effects are demonstrated thereon.

The following provides a more detailed explanation of the present invention through examples and test examples, but the present invention is not limited thereto.

### EXAMPLES

The following provides an explanation in accordance with the following examples.

### Example 1

Kerosene was added to 0.4 g of the subject compound to prepare an oily undiluted aerosol liquid (120 mL) followed by filling into an aerosol container. After attaching a valve, 180 mL of a propellant (mixed gas of liquefied petroleum gas and dimethyl ether) were filled through the valve under pressure to obtain an aerosol insecticide for flying insects.

When this aerosol insecticide was sprayed for 2 seconds at house flies present in a living room, the house flies were immediately knocked down and did not revive.

### Example 2

8.0 g of a surfactant, 30 mL of water and kerosene were added to 0.35 g of the subject compound to prepare an aqueous undiluted aerosol liquid (120 mL) followed by filling into an aerosol container. After attaching a valve, 180 mL of a propellant (liquefied petroleum gas) was filled through the valve under pressure to obtain an aerosol insecticide for controlling crawling insects targeted at cockroaches.

This aerosol insecticide demonstrated high knockdown effects and lethal effects against cockroaches, and in the case of residual treatment of locations where cockroaches are present, superior residual efficacy was demonstrated over the course of several weeks.

### Example 3

2-Methyl-4-oxo-3-(2-propynyl)-2-cyclopenten-1-yl 2,2-dimethyl-3-(2,2-dichlorovinyl)cyclopropanecarboxylate has a total of eight isomers based on two asymmetric carbon atoms of the acid portion and one asymmetric carbon atom of the alcohol portion. Therefore, a comparative efficacy test was conducted on female house flies using the microdrop method for the subject compound and the other seven isomers. Those results are shown in Table 1.

TABLE 1

| | | | Insecticidal Efficacy | |
|---|---|---|---|---|
| | Isomer | | LD₅₀ | Relative |
| Compound No. | Alcohol 1) | Acid 2) | (μg/insect) | efficacy ratio |
| 1 (Subject compound) | d | d-trans | 0.0263 | 100 |
| 2 | d | d-cis | 0.0415 | 63.0 |
| 3 | l | d-trans | 0.245 | 11.0 |
| 4 | l | d-cis | 0.376 | 7.0 |
| 5 | d | 1-trans | 0.337 | 7.8 |
| 6 | d | 1-cis | 0.526 | 5.0 |
| 7 | l | 1-trans | 3.29 | 0.8 |
| 8 | l | 1-cis | 5.30 | 0.5 |

1): The d-forms and l-forms respectively correspond to the (S)-forms and (R)-forms of the absolute configuration.
2): The d-trans form, d-cis form, l-trans form and l-cis form respectively correspond to the (1R)-trans form, (1R)-cis form, (1S)-trans form and (1S)-cis form of the absolute configuration.

As a result of the test, the isomer of the subject compound was confirmed to have remarkably higher insecticidal efficacy than the other seven isomers.

In contrast, although Japanese Patent Publication No. Sho 61-57820 (Patent Document 1) discloses that various mixtures of these isomers have high knockdown effects ((1) mixture of eight compounds consisting of Compounds 1 to 8, (2) mixture of four compounds consisting of Compounds 1, 3, 5 and 7, (3) mixture of four compounds consisting of Compounds 2, 4, 6 and 8, and (4) mixture of two compounds consisting of Compounds 2 and 6), this publication merely refers to the efficacy of mixtures, and the insecticidal efficacy of the subject compound per se cannot be derived by calculation of these. Namely, in Patent Document 1, the subject compound is completely omitted and Patent Document 1 cannot be said to be a document that suggests the subject compound.

According to Non-Patent Document 3, the relative efficacy ratios against house flies using as determined using the microdrop method are reported as shown in Table 2 for eight isomers of prallethrin (2-methyl-4-oxo-3-(2-propynyl)-2-cyclopenten-1-yl 2,2-dimethyl-3-(2-methyl-1-propenyl)cyclopropanecarboxylate), which is different from the subject compound only in the acid portion.

TABLE 2

| | Isomer | | Relative efficacy |
|---|---|---|---|
| Compound No. | Alcohol | Acid | ratio |
| 1 | d | d-trans | 100 |
| 2 | d | d-cis | 39 |
| 3 | l | d-trans | 17 |
| 4 | l | d-cis | 13 |
| 5 | d | 1-trans | 1.3 |
| 6 | d | 1-cis | 0.4 |
| 7 | l | 1-trans | 0.2 |
| 8 | l | 1-cis | 0.2 |

A comparison of Tables 1 and 2 revealed that isomers in which the acid portion is an ester of the d-trans form demonstrate superior insecticidal efficacy as compared with those of the d-cis form for the subject compound and all of the isomers of prallethrin, and that the relationship between three-dimensional structure and insecticidal efficacy demonstrated a similar trend for all eight isomers.

These results clearly conflict with the general theory derived from Non-Patent Document 1 as relating to permethrin, namely the conventional technical idea that among esters of 2,2-dimethyl-3-(2,2-dicyclovinyl)cyclopropanecarboxylic acid, esters of acids of the d-cis form have superior insecticidal efficacy to esters of acids of the d-trans form.

Since the results in Table 2 relating to prallethrin do not necessarily suggest superior efficacy as in Table 1 for the subject compound, and it is necessary to verify insecticidal efficacy after actually tested each isomer as in Table 1, the present invention was completed on the basis of evidence from actual test data.

### Example 4

A test was next conducted of the efficacy of test compounds in the form of aerosols.

### (1) Test Against House Flies

Glass chamber method: test aerosol insecticides prepared in accordance with Example 1 were sprayed for 1 second in a glass chamber having a volume of 60×60×60 cm (0.216 m³). House flies (in groups of about 25 female adults each) were released into the chamber 10 seconds later, and the numbers of test mosquitoes that fell to the ground and rolled over during 10 minutes of exposure were recorded over the course of time to determine the KT₅₀ values followed by observation of the kill rate after an additional 24 hours. Those results are shown in Table 3.

TABLE 3

| | Test Aerosol | | | |
|---|---|---|---|---|
| | Compound | g/300 ml | KT₅₀ value (sec) | Mortality (%) |
| 1 | Subject compound | 0.45 | 36 | 100 |
| 2 | Subject compound | 0.15 | 73 | 93.3 |
| 3 | d-T80-phthalthrin | 0.45 | 73 | 39.2 |
| 4 | d-T80-phthalthrin | 0.15 | 204 | 20.0 |

According to the results of this test, the subject compound exhibited rapid action roughly 2 to 3 times greater than the d-T80-phthalthrin, conventionally considered to be the most superior knockdown agent of the prior art. In addition, lethal effects were also extremely high, thereby confirming that the subject compound is able to provide a practical and economical aerosol insecticide provided with both rapid action and lethal effects.

### (2) Test Against Cockroaches

(2-a) Turntable Method: A glass cylinder having an inner diameter of 20 cm and height of 43 cm was placed on a wooden stand and a glass pot (outer diameter: 15 cm, height: 15 cm) containing two adult male American cockroaches was attached directly beneath the cylinder by using a glass plate as a shutter. Test aerosol insecticides prepared in accordance with Example 1 were sprayed into the glass cylinder for 1 second. Five seconds later, the glass plate shutter was pulled out and the test cockroaches were exposed to the descending aerosol particles for 5 minutes. The range of times until the occurrence of knockdown and the approximate average times were determined followed by observation of mortality 24 hours later. This test was repeated six times. The results are shown in Table 4.

(2-b) Residual Contact Method: The test aerosols were sprayed onto a decorative sheet measuring 20 cm on a side at the rate of 25 mL/m². After 1 day of spraying, a glass ring measuring 12 cm in diameter was placed on the decorative sheet and five American cockroaches were contacted with the sheet for 2 hours inside the ring at 1, 7, 15 and 21 days after spraying followed by observation of mortality 72 hours later. Those results are shown in Table 5.

TABLE 4

| | | | | Mortality |
|---|---|---|---|---|
| | Test Aerosol | | KT₅₀ value | after 24 |
| | Compound | g/300 ml | (sec) | hours (%) |
| 1 | Subject compound | 0.35 | 102 | 100 |
| 2 | Imiprothrin | 0.35 | 51 | 83 |
| 3 | d-T80-phthalthrin | 0.35 | >300 | 33 |

TABLE 5

| | | | Mortality after 72 hours (%) | | | |
|---|---|---|---|---|---|---|
| | Test Aerosol | | After 1 day | After 7 | After 15 | After 21 |
| | Compound | g/300 ml | of spray | days | days | days |
| 1 | Subject | 0.35 | 100 | 100 | 100 | 100 |
| | compound | | | | | |

| | | | | |
|---|---|---|---|---|
| 2 | Imiprothrin | 0.35 | 0 | — |

As a result of tests on cockroaches using the turntable method, the subject compound demonstrated rapid action remarkably greater than d-T80-phthalthrin although not to the extent of imiprothrin, while also having extremely high lethal effects.

On the other hand, as a result of testing using the residual contact method, in contrast to imiprothrin being observed to be completely absent of residual efficacy, with mortality of 0% at one day after spraying, the subject compound maintained mortality of 100% up to 21 days after spraying. One reason for the complete absence of residual efficacy for imiprothrin can be presumed to be the physical property of imiprothrin in the form of its extremely poor solubility in kerosene.

Conversely, the subject compound has satisfactory compatibility with solvents such as kerosene, and not only demonstrates superior function in direct spraying treatment, but also was confirmed to be an extremely useful active ingredient in terms of residual treatment as well.

The home-use insecticide of the present invention contains a pyrethroid compound having superior insecticidal activity and is highly safe with respect to warm-blooded animals as a knockdown agent and killing agent for harmful insects. Thus, since it is provided with even better rapid action as well as superior lethal effects and residual efficacy as compared with current products against various flying insects and crawling insects, it has an extremely high degree of practicality.

The present invention can be used for the purpose of controlling a wide range of harmful insects both indoors and outdoors.

What is claimed is:

1. A home-use insecticide comprising (d)-2-methyl-4-oxo-3-(2-propynyl)-2-cyclopenten-1-yl (d)-trans-2,2-dimethyl-3-(2,2-dichlorovinyl)cyclopropanecarboxylate as an active ingredient thereof.

2. The home-use insecticide according to claim 1, wherein the active ingredient is effective as both an insect knockdown agent and an insect killing agent.

3. The home-use insecticide according to claim 1, wherein the insecticide is in the form of an aerosol.
